(19) United States
(12) Patent Application Publication
(10) Pub. No.: US 2022/0031884 A1
(43) Pub. Date: Feb. 3, 2022

(54) ULTRAVIOLET LED SANITIZING CABINET

(71) Applicant: LiteIdeas, LLC, Forest, VA (US)

(72) Inventor: Roger Whyte, Charlottesville, VA (US)

(21) Appl. No.: 17/443,996

(22) Filed: Jul. 29, 2021

Related U.S. Application Data
(60) Provisional application No. 62/706,059, filed on Jul. 29, 2020.

(51) Int. Cl. A61L 2/10; F21V 7/22

(52) U.S. Cl. CPC A61L 2/10; F21Y 2115/10; F21V 7/22

(57) ABSTRACT

A system includes a cabinet having an enclosed cavity having a plurality of reflective interior walls that reflect UV radiation and a plurality of UV-emitting LEDs. The plurality of LEDs are arranged on at least four of the reflective interior walls of the enclosed cavity and configured to direct UV radiation into an interior of the cavity.

### CROSS-REFERENCE TO RELATED APPLICATIONS

This application claims the benefit of U.S. Provisional Application No. 62/706,059, filed Jul. 29, 2020, the disclosure of which is incorporated herein by reference in its entirety.

### TECHNICAL FIELD

This document relates to sanitizing objects with ultraviolet light and, in particular, to an ultraviolet LED sanitizing cabinet.

### BACKGROUND

The risk of exposure to pathogens, such as, for example, viruses, bacteria, and the like, is high, and a need exists to quickly, easily, and economically kill such pathogens on surfaces to which people are exposed.

### SUMMARY

In a general aspect, a system includes a cabinet having an enclosed cavity having a plurality of reflective interior walls that reflect UV radiation and

a plurality of UV-emitting LEDs. The plurality of LEDs are arranged on at least four of the reflective interior walls of the enclosed cavity and configured to direct UV radiation into an interior of the cavity.

Implementations can include one or more of the following features, alone or in any combination with each other.

For example, the plurality of LEDs can include at least 20 LEDs.

In another example, a power of UV radiation emitted from each of the LEDs can be greater than 5 mW.

In another example, the enclosed cavity can include corner panels at each intersection of adjacent ones of the interior walls.

In another example, at least two of the plurality of LEDs can be arranged on each of the corner panels.

In another example, the cavity can include a wire rack configured for supporting an object while UV radiation is directed from the LEDs into the interior of the cavity.

In another example, the system can further include a vibrator coupled to the wire rack and configured to vibrate the wire rack while an object is supported on the wire rack and UV radiation is directed into the interior of the cavity.

In another example, the cavity can include a hook configured for supporting an object suspended from the hook.

In another example, the system can further include a vibrator coupled to the hook and configured to vibrate the hook while an object is suspended from the hook and UV radiation is directed into the interior of the cavity.

In another example, an angular intensity distribution of the LEDs can be greater than 100 degrees at a full-width, half-maximum of the distribution.

In another example, a difference between a local intensity maximum and a local intensity maximum of the light field emitted by the plurality of UV-emitting LEDs can be less than the 50% of the local intensity maximum.

In another example, the system can further include a tag reader configured to read tags that uniquely identify objects placed within the cabinet.

In another general aspect, a method of sanitizing an object includes, when an object is placed within a cabinet equipped with a plurality of UV LEDs, providing UV radiation from the plurality of UV LEDs to the object, locking a door to the cabinet while the UV radiation is provided from the UV LEDs to the object, and controlling a total power of UV radiation provided from the UV LEDs to the object and a time duration during which the UV radiation is provided to the object such that a total UV energy provided to a surface of the object exceeds a UV energy that kills more than 99.99% of a predetermined pathogen on the surface of the object.

Implementations can include one or more of the following features, alone or in any combination with each other.

For example, the method can further include vibrating a support for the object that is placed within the cabinet while the UV radiation is provided to the object.

In another example, the method can further include reading, with a tag reader, a unique identifier associated with the object while the object is placed within the cabinet.

In another example, providing the UV radiation includes providing the UV radiation such that a difference between a local intensity maximum and a local intensity maximum of the light field emitted by the plurality of UV-emitting LEDs is less than the 50% of the local intensity maximum.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

FIG. 1 is a block diagram illustrating a portion of a sanitizing system that includes an enclosed cabinet into which objects can be placed for sanitizing/decontamination by UV light.

FIG. 2 is a perspective view of an example implementation of an interior cavity of the cabinet of FIG. 1.

FIG. 3 is a graph of an example angular distribution of UV radiation from UV-emitting LED, in which the full-width, half-maximum intensity spans about 110 degrees, or about 55 degrees on either side of a zero angle axis of light emitted from the LED.

FIG. 4 is a perspective view of an another example implementation of an interior cavity of the cabinet of FIG. 1.

The components in the drawings are not necessarily drawn to scale and may not be in scale relative to each other. Like reference numerals designate corresponding parts throughout the several views.

### DETAILED DESCRIPTION

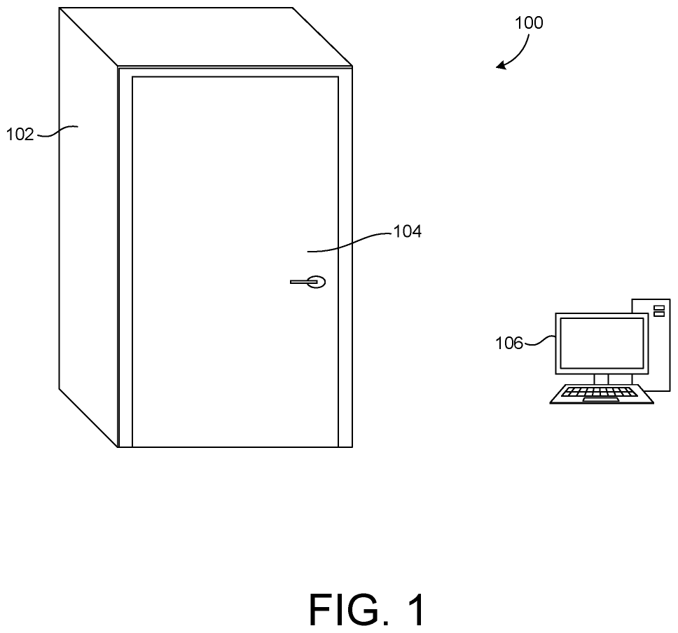

FIG. 1 is a block diagram illustrating a portion of a sanitizing system 100 that includes an enclosed cabinet 102 into which objects can be placed for sanitizing/decontamination by ultraviolet (UV) light. The cabinet 102 is configured to decontaminate contents from harmful viruses and bacteria in a fast, clean, environmentally-friendly, and low cost manner.

The cabinet 102 can include a door 104 through which access to an interior cavity of the cabinet can be gained. The components of the cabinet 102 can be controlled by a computer and/or processor 106 that executes machine-readable instructions stored in a memory device. In some implementations, the computer and/or processor 106 can be integrated into the cabinet 102. In some implementations, the computer and/or processor 106 can be separate from the cabinet 102 and can communicate with the cabinet over a wired and/or wired connection.

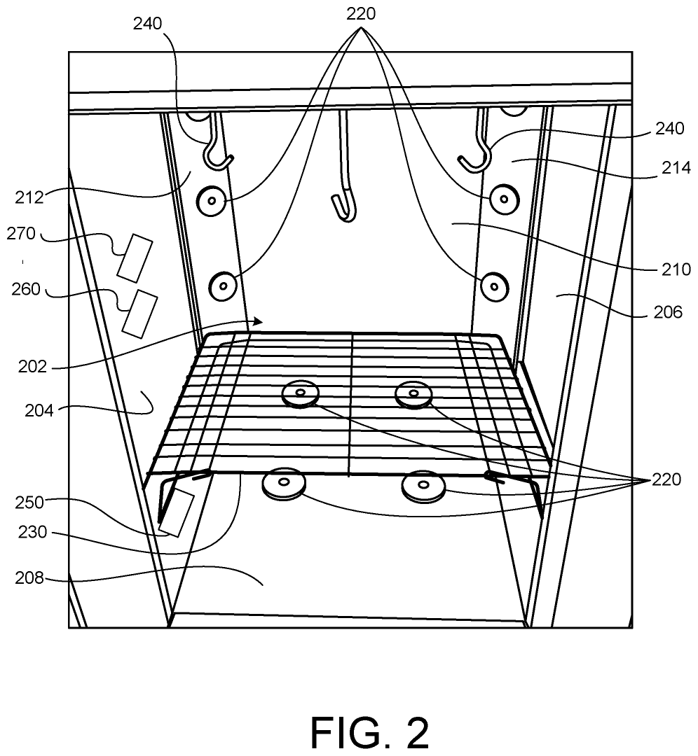

FIG. 2 illustrates an example interior cavity 202 of the cabinet 102. In some implementations, the interior cavity 202 can have a generally rectilinear shape defined by six main walls. For example, in some implementations, the six main walls can include a left wall 204, a right wall 206, a bottom wall 208, a top wall (not shown), a back wall 210, and a front wall (not shown). In some implementations, the left wall 204 can be generally parallel to, and generally the same size and shape as, the right wall 206; the bottom wall 208 can be generally parallel to, and generally the same size and shape as, the top wall; and the back wall 210 can be generally parallel to, and generally the same size and shape as, the front wall. Thus, the six main walls can define a rectilinear solid volume within the cabinet 102, where adjacent main walls are oriented at approximately ninety-degree angles to each other.

In some implementations, the cavity 202 of the cabinet 102 can be further defined by generally rectangular corner panels 212, 214 located at joints between the adjacent side walls 204, 206, the back wall 210 and the front wall. In some implementations, each of the corner panels 212, 214 can be arranged at forty-five degree angles to its adjacent sidewalls 204, 206, 210. The corner panels 212, 2214 can be rectangular in shape and can extend along a vertical length of the cavity 202 at the intersection of adjacent sidewalls 204, 206, 210.

A plurality of UV-emitting light emitting diodes (LEDs) 220 can be placed in walls 204, 206, 208, 210, and/or corner panels 212, 214 and configured to direct UV radiation into the cavity 202 to create a three-dimensional intensity field of UV light (a light field) within the cavity 202. For example, in one implementation, four UV-emitting LEDs arranged in a rectangular pattern and can be located in each of the bottom wall 208 and the top wall that define the cavity 202, and three UV-emitting LEDs can be placed in each of the corner panels 212, 214 that define the cavity 202, such that the a total of a least 20 UV-emitting LEDs direct light into the cavity 202.

In some implementations, the four UV-emitting LEDs in each of the bottom wall 208 and the top wall can be arranged such that an area enclosed by the rectangle having vertices at the locations of the UV-emitting LEDs is greater than 16% of an area of the wall in which the UV-emitting LEDs are located. Thus, for example, in a 10″×10″ wall, the UV-emitting LEDs can be arranged in a square pattern having sides with lengths greater than 4″. In some implementations, the four UV-emitting LEDs in each of the bottom wall 208 and the top wall can be arranged such that an area enclosed by the rectangle having vertices at the locations of the UV-emitting LEDs is greater than 25% of an area of the wall in which the UV-emitting LEDs are located. Thus, for example, in a 10″×10″ wall, the UV-emitting LEDs can be arranged in a square pattern having sides with lengths greater than 5″. In some implementations, the four UV-emitting LEDs in each of the bottom wall 208 and the top wall can be arranged such that an area enclosed by the rectangle having vertices at the locations of the UV-emitting LEDs is greater than 35% of an area of the wall in which the UV-emitting LEDs are located. Thus, for example, in a 10″×10″ wall, the UV-emitting LEDs can be arranged in a square pattern having sides with lengths greater than 5.9″. More or fewer than four UV-emitting LEDs can be located in the side, top, and or bottom walls.

In some implementations, three UV-emitting LEDs 220 in the corner panels 212, 214, and the UV-emitting LEDs can be equally spaced along a length of each corner panel. For example, in a 15″ long corner panel, UV-emitting LEDs can be placed at the 3.75″, 7.5″ and 11.25″ along the length of the panel. More or fewer than three UV-emitting LEDs can be located in the corner panels.

The UV-emitting LEDs 220 can emit ultraviolet light, for example, light having a spectrum with a peak intensity less than 350 nm. In some implementations, the peak intensity wavelength of the UV-emitting LEDs can be approximately 275 nm.

The interior walls of the cavity 202 can include material (e.g., aluminum, stainless steel, etc.) that is highly reflective (e.g., with a reflectivity of greater than 90%) of the spectrum of light emitted by the UV-emitting LEDs 220. A lens may be placed over an LED to define an angular emission distribution of the light emitted from the LED. The angular emission distribution of the UV-emitting LEDs 220 can be selected to emit light in a distribution pattern that spreads light throughout the cavity 202.

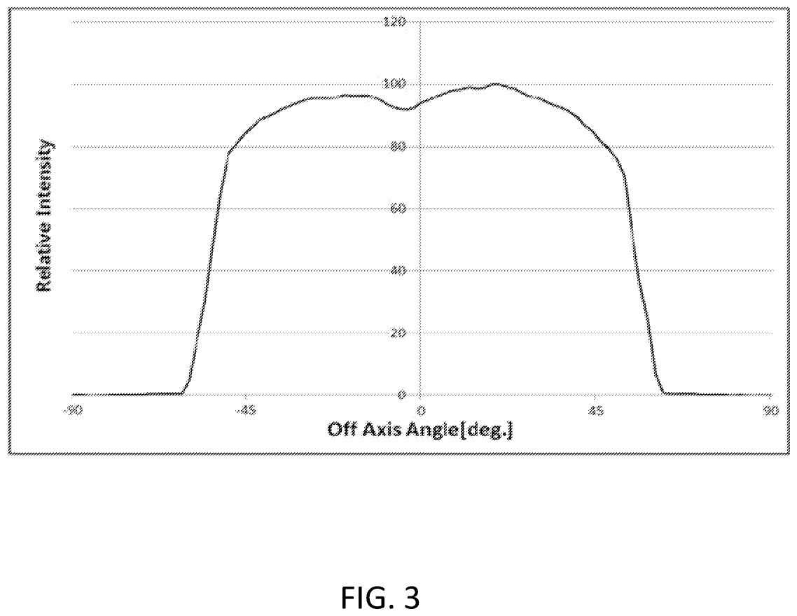

FIG. 3 is a graph of an example angular distribution of UV radiation from a UV-emitting LED 220, in which the full-width, half-maximum intensity spans about 110 degrees, or about 55 degrees on either side of a zero angle axis of light emitted from the LED. The geometry of the cavity 202, the placement of the LEDs, and the angular intensity distribution of the LEDs can be selected such that, within a volume that is 70% of the total volume of the cavity and includes no point that is less than 4% of a respective width, length, or height of the cavity away from a wall from which the respective width, length, or height is defined, a difference between a local intensity maximum and a local intensity maximum of the light field emitted by the plurality of UV-emitting LEDs can be less than the 50% of the local intensity maximum. For example, in a cubic cavity having sides of length, a, within a cubic volume of sides 0.9a and with each wall of the cubic volume being spaced more than 0.02a from a wall of the cavity, the difference between a local intensity maximum and a local intensity maximum of the light field emitted by the plurality of UV-emitting LEDs 220 can be less than the 50% of the local intensity maximum.

The radiant flux of UV radiation from the UV-emitting LEDs 220 in the cavity 202 can be selected, so that the local intensity is sufficient to kill a threshold percentage of microorganisms (e.g., bacteria, viruses, etc.) within a threshold amount of time. For example, in a cavity 202 having a volume of approximately 0.15 cubic meters that is irradiated by 20 UV-emitting LEDs 220 (four located in a square pattern in each of the top and bottom walls and three located in each of the corner panels), each producing UV radiation power of at least 5 milliwatts or at least 7 milliwatts, the UV radiation intensity in the cavity can be sufficient to kill more than 99.99% of poliovirus, influenza virus, or staphylococcus within five minutes, or 99.99% of salmonella typhimurium within 10 minutes. Thus, the UV light field in the cavity 202 of the cabinet can be an effective tool for sanitizing or decontaminating surfaces of dangerous pathogens.

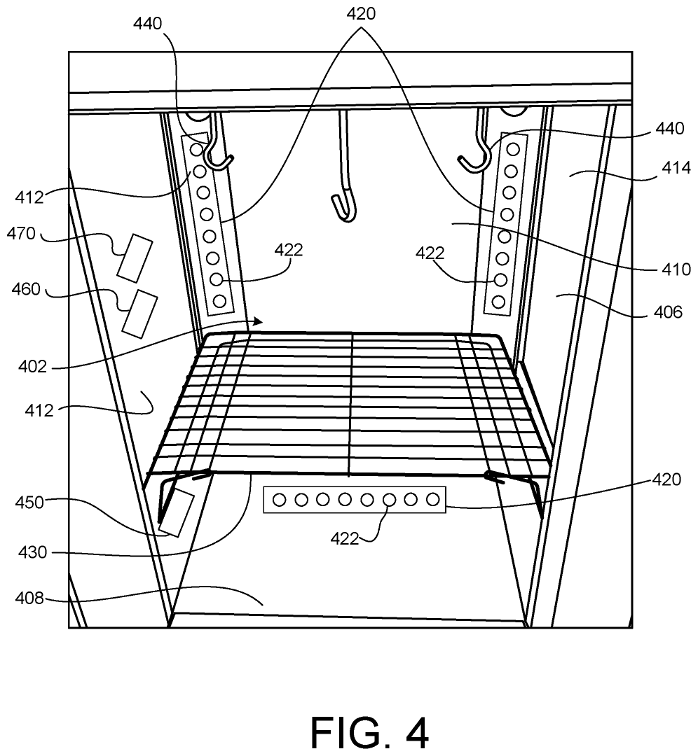

FIG. 4 is a perspective view of another example implementation of an interior cavity 402 of the cabinet of FIG. 1. The interior cavity 402 can have a generally rectilinear shape defined by six main walls that can include a left wall 404, a right wall 406, a bottom wall 408, a top wall (not shown), a back wall 410, and a front wall (not shown). In some implementations, the left wall 404 can be generally parallel to, and generally the same size and shape as, the right wall 406; the bottom wall 408 can be generally parallel to, and generally the same size and shape as, the top wall; and the back wall 410 can be generally parallel to, and generally the same size and shape as, the front wall. Thus, the six main walls can define a rectilinear solid volume within the cabinet 102, where adjacent main walls are oriented at approximately ninety-degree angles to each other.

In some implementations, the cavity 402 of the cabinet 102 can be further defined by generally rectangular corner panels 412, 414 located at joints between the adjacent side walls 404, 406, the back wall 410 and the front wall. In some implementations, each of the corner panels 412, 414 can be arranged at forty-five degree angles to its adjacent sidewalls 404, 406, 410. The corner panels 412, 4214 can be rectangular in shape and can extend along a vertical length of the cavity 402 at the intersection of adjacent sidewalls 404, 406, 410.

A plurality of UV-emitting light emitting diode (LED) modules 420 that each include a plurality of LEDs 422 can be placed in walls 404, 406, 408, 410, and/or corner panels 412, 414 and configured to direct UV radiation into the cavity 402 to create a three-dimensional intensity field of UV light (a light field) within the cavity 402. Each module 420 can include a plurality (e.g., five or more, ten or more, twenty or more) of individual LEDs 422. For example, in one implementation, a UV-emitting LED module 420 having individual LEDs 422 arranged in a rectangular pattern can be located in each of the bottom wall 408 and the top wall that define the cavity 402, and three such UV-emitting LED modules can be placed in each of the corner panels 412, 414 that define the cavity 402.

The UV-emitting LEDs 422 can emit ultraviolet light, for example, light having a spectrum with a peak intensity less than 350 nm. In some implementations, the peak intensity wavelength of the UV-emitting LEDs can be approximately 275 nm.

The interior walls of the cavity 402 can include material (e.g., aluminum, stainless steel, etc.) that is highly reflective (e.g., with a reflectivity of greater than 90%) of the spectrum of light emitted by the UV-emitting LEDs 422. A lens may be placed over an LED to define an angular emission distribution of the light emitted from the LED. The angular emission distribution of the UV-emitting LEDs 422 can be selected to emit light in a distribution pattern that spreads light throughout the cavity 402. With a high density of LEDs 422 in the LED modules 420, a high power density of UV light in the cavity 402 can be achieved. For example,

Objects to be sanitized or decontaminated can be placed in the cabinet and located with the cavity in positions where the UV light filed is relatively uniform (e.g., is free of shadows and minima in the light field intensity). For example, referring again to FIG. 2 and to FIG. 4, the cabinet 102 can include one or more wire racks 230, 430 on which objects may be placed and/or hooks 240, 440 on which objects may be suspended.

Wire racks 230, 430 can include a plurality of parallel rods or wires, which can include, for example, metal, plastic, or composite material. The rods or wires can have a curved (e.g., circular, oval, elliptical) cross section, such that a hard object placed on the rods or wires of the racks 230, 430, contacts only a small surface area of the wire rack, and therefore UV light that is either directly supplied from a UV LED or indirectly supplied from a UV LED (e.g., after being reflected by one or more surfaces within the cavity 202, 402) can reach all, or nearly all of the surface area of the object. Similarly, hooks 240, 440 can have a curved (e.g., circular, oval, elliptical) cross section, such that a hard object suspended from a hook 240, 440 contacts only a small surface area of the hook, and therefore UV light that is either directly supplied from a UV LED or indirectly supplied from a UV LED (e.g., after being reflected by one or more surfaces within the cavity 202, 402) can reach all, or nearly all of the surface area of the object.

In another implementation, a wire rack 230, 430 and/or a hook 240, 440 can be mechanically coupled to a vibrator (e.g., a piezoelectric device, a motor, or an oscillator) 250, 450 that is configured to vibrate the wire rack 230, 430 and/or the hook 240, 440 to cause an object placed on the wire rack 230, 430 or suspended from the hook to vibrate or “jiggle” with respect to the wire rack or hook while it is placed on the wire rack or suspended from the hook. Because of the vibration of the object with respect the wire rack or the hook that support the object, the points at which the object is supported by the wire race or the hook can vary over the time at which UV light is supplied to the object and/or the points at which the object is supported may not be in contact with the wire rack or the hook for small time periods, such that all points of the surface of the object are exposed to the UV light, at least temporarily, during the time during which UV light is supplied within the cabinet with the object in the cabinet.

In another implementation, the cavity 202, 402 can include a UV dosimeter 260, 460 that measures a total dose of UV energy received by the dosimeter over a time period in which UV light is supplied by the LEDs in the cavity 202, 402 while the object is inside the cavity.

In another implementation, the cavity 202, 402 can include a tag reader 270, 470 (e.g., an RFID tag reader, an optical scanner, a QR code reader, etc.) that is configured to read a unique identifier associated with an object that is placed within the cavity. The tag reader 270, 470 can identify an object placed within the cavity and irradiated by UV light and then can communicate a message to a database to indicate that the object has been irradiated by the UV light.

The operation of the cabinet 102 can be operated under computer control by computer or processor 106. For example, the computer or processor can program and control a door interlock to lock the door 104 and to prevent the door 104 from being unlocked while UV LEDs 220, 422 are turned on within the cabinet 102. In addition, the computer 106 or processor can program the intensity of the LEDs 220 and the duration of irradiation from the LEDs in the cavity 202, 402 of the cabinet 102 to achieve a desired UV light power within the cavity 202, 402. In another implementation, the computer 106 or processor can receive signals and information from the dosimeter 260, 460 and from the tag reader 270, 470, which can be used to determine that a particular object uniquely identified by a code read by the tag reader was inside the cavity 202, 402 and received a particular dose of UV light at a particular date and time. Such information can be communicated from the computer or processor 106 to a database, which maintains information about the object and its status whether or not it is, at a particular time, sanitized by virtue of having been exposed to UV light in the cabinet.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. Various implementations of the systems and techniques described here can be realized as and/or generally be referred to herein as a circuit, a module, a block, or a system that can combine software and hardware aspects. For example, a module may include the functions/acts/computer program instructions executing on a processor (e.g., a processor formed on a silicon substrate, a GaAs substrate, and the like) or some other programmable data processing apparatus.

Some of the above example implementations are described as processes or methods depicted as flowcharts. Although the flowcharts describe the operations as sequential processes, many of the operations may be performed in parallel, concurrently, or simultaneously. In addition, the order of operations may be re-arranged. The processes may be terminated when their operations are completed but may also have additional steps not included in the figure. The processes may correspond to methods, functions, procedures, subroutines, subprograms, etc.

Methods discussed above, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a storage medium. A processor(s) may perform the necessary tasks.

Specific structural and functional details disclosed herein are merely representative for purposes of describing example implementations. Example implementations, however, be embodied in many alternate forms and should not be construed as limited to only the implementations set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example implementations. As used herein, the term and/or includes any and all combinations of one or more of the associated listed items.

It will be understood that when an element is referred to as being connected or coupled to another element, it can be directly connected or coupled to the other element or intervening elements may be present. In contrast, when an element is referred to as being directly connected or directly coupled to another element, there are no intervening elements present. Other words used to describe the relationship between elements should be interpreted in a like fashion (e.g., between versus directly between, adjacent versus directly adjacent, etc.).

The terminology used herein is for the purpose of describing particular implementations only and is not intended to be limiting of example implementations. As used herein, the singular forms a, an and the are intended to include the plural forms as well, unless the context clearly indicates otherwise. It will be further understood that the terms comprises, comprising, includes and/or including, when used herein, specify the presence of stated features, integers, steps, operations, elements and/or components, but do not preclude the presence or addition of one or more other features, integers, steps, operations, elements, components and/or groups thereof.

It should also be noted that in some alternative implementations, the functions/acts noted may occur out of the order noted in the figures. For example, two figures shown in succession may in fact be executed concurrently or may sometimes be executed in the reverse order, depending upon the functionality/acts involved.

Unless otherwise defined, all terms (including technical and scientific terms) used herein have the same meaning as commonly understood by one of ordinary skill in the art to which example implementations belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the above example implementations and corresponding detailed description are presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the above illustrative implementations, reference to acts and symbolic representations of operations (e.g., in the form of flowcharts) that may be implemented as program modules or functional processes include routines, programs, objects, components, data structures, etc., that perform particular tasks or implement particular abstract data types and may be described and/or implemented using existing hardware at existing structural elements. Such existing hardware may include one or more Central Processing Units (CPUs), digital signal processors (DSPs), application-specific-integrated-circuits, field programmable gate arrays (FPGAs) computers or the like.

It should be borne in mind, however, that all of these and similar terms are to be associated with the appropriate physical quantities and are merely convenient labels applied to these quantities. Unless specifically stated otherwise, or as is apparent from the discussion, terms such as processing or computing or calculating or determining of displaying or the like, refer to the action and processes of a computer system, or similar electronic computing device, that manipulates and transforms data represented as physical, electronic quantities within the computer system's registers and memories into other data similarly represented as physical quantities within the computer system memories or registers or other such information storage, transmission or display devices.

Note also that the software implemented aspects of the example implementations are typically encoded on some form of non-transitory program storage medium or implemented over some type of transmission medium. The program storage medium may be magnetic (e.g., a floppy disk or a hard drive) or optical (e.g., a compact disk read only memory, or CD ROM), and may be read only or random access. Similarly, the transmission medium may be twisted wire pairs, coaxial cable, optical fiber, or some other suitable transmission medium known to the art. Example embodiments are not limited by these aspects of any given implementation.

While example embodiments may include various modifications and alternative forms, embodiments thereof are shown by way of example in the drawings and will herein be described in detail. It should be understood, however, that there is no intent to limit example embodiments to the particular forms disclosed, but on the contrary, example embodiments are to cover all modifications, equivalents, and alternatives falling within the scope of the claims.

What is claimed is:

1. A system comprising:
a cabinet having an enclosed cavity having a plurality of reflective interior walls that reflect UV radiation; and
a plurality of UV-emitting LEDs, wherein
the plurality of LEDs are arranged on at least four of the reflective interior walls of the enclosed cavity and configured to direct UV radiation into an interior of the cavity.

2. The system of claim 1, wherein the plurality of LEDs includes at least 20 LEDs.

3. The system of claim 1, wherein a power of UV radiation emitted from each of the LEDs is greater than 5 mW.

4. The system of claim 1, wherein the enclosed cavity includes corner panels at each intersection of adjacent ones of the interior walls.

5. The system of claim 4, wherein at least two of the plurality of LEDs are arranged on each of the corner panels.

6. The system of claim 1, wherein the cavity includes a wire rack configured for supporting an object while UV radiation is directed from the LEDs into the interior of the cavity.

7. The system of claim 6, further comprising a vibrator coupled to the wire rack and configured to vibrate the wire rack while an object is supported on the wire rack and UV radiation is directed into the interior of the cavity.

8. The system of claim 1, wherein the cavity includes a hook configured for supporting an object suspended from the hook.

9. The system of claim 8, further comprising a vibrator coupled to the hook and configured to vibrate the hook while an object is suspended from the hook and UV radiation is directed into the interior of the cavity.

10. The system of claim 1, where an angular intensity distribution of the LEDs is greater than 100 degrees at a full-width, half-maximum of the distribution.

11. The system of claim 1, wherein a difference between a local intensity maximum and a local intensity maximum of a light field emitted by the plurality of UV-emitting LEDs is less than the 50% of the local intensity maximum.

12. The system of claim 1, further comprising a tag reader configured to read tags that uniquely identify objects placed within the cabinet.

13. A method of sanitizing an object, the method comprising:
when an object is placed within a cabinet having a plurality of UV-emitting LEDs, providing UV radiation from the plurality of UV LEDs to the object;
locking a door to the cabinet while the UV radiation is provided from the UV LEDs to the object; and
controlling a total power of UV radiation provided from the UV LEDs to the object and a time duration during which the UV radiation is provided to the object such that a total UV energy provided to a surface of the object exceeds a UV energy that kills more than 99.99% of a predetermined pathogen on the surface of the object.

14. The method of claim 13, further comprising:
vibrating a support for the object that is placed within the cabinet while the UV radiation is provided to the object.

15. The method of claim 13, further comprising:
reading, with a tag reader, a unique identifier associated with the object while the object is placed within the cabinet.

16. The method of claim 13, wherein providing the UV radiation includes providing the UV radiation such that a difference between a local intensity maximum and a local intensity maximum of a light field emitted by the plurality of UV-emitting LEDs is less than the 50% of the local intensity maximum.
